(19) United States
(12) Patent Application Publication
(10) Pub. No.: US 2019/0175001 A1
(43) Pub. Date: Jun. 13, 2019

(54) ENDOSCOPE

(71) Applicant: OLYMPUS CORPORATION, Tokyo (JP)

(72) Inventor: Yuta SEKIGUCHI, Tokyo (JP)

(73) Assignee: OLYMPUS CORPORATION, Tokyo (JP)

(21) Appl. No.: 16/273,222

(22) Filed: Feb. 12, 2019

Related U.S. Application Data
(63) Continuation of application No. PCT/JP2017/031178, filed on Aug. 30, 2017.

(30) Foreign Application Priority Data
Oct. 24, 2016 (JP) 2016-207819

(51) Int. Cl. A61B 1/05; A61B 1/00; G02B 23/26

(52) U.S. Cl. CPC A61B 1/05; A61B 1/00096; A61B 1/0661; G02B 23/26; A61B 1/00121; A61B 1/00163

(57) ABSTRACT

An endoscope is provided with: a first frame body turnably provided around shaft bodies, the first frame body holding an optical device; a second frame body turnably holding the first frame body; recessedly formed groove portions, the groove portions being in contact with outer circumferential faces of the shaft bodies; and an elastic member configured to give a tensile load in a direction in which the shaft bodies and the groove portions are in contact.

### CROSS REFERENCE TO RELATED APPLICATION

This application is a continuation application of PCT/JP2017/031178 filed on Aug. 30, 2017 and claims benefit of Japanese Application No. 2016-207819 filed in Japan on Oct. 24, 2016, the entire contents of which are incorporated herein by this reference.

### BACKGROUND OF INVENTION

### 1. Field of the Invention

The present invention relates to a variable direction-of-view endoscope, and in particular to an endoscope in which a direction of view is changed by moving an optical device provided on a distal end portion of an insertion portion.

### 2. Description of the Related Art

In order to observe a position where observation is difficult, such as an inside of a living body or an inside of a structure, endoscopes for picking up an optical image, which can be introduced into the inside of the living body or the structure from outside, are used, for example, in a medical field or an industrial field.

Among the endoscopes, there are endoscopes having a flexible insertion portion, which are used for examination and treatment of digestive tracts, and endoscopes having a rigid insertion portion, which are used for surgical operations.

Especially, the endoscopes having a rigid insertion portion is referred to as a rigid endoscope, a laparoscope, a pyeloscope and the like, and, for example, as disclosed in Japanese Patent Application Laid-Open Publication No. 07-327916, a variable direction-of-view endoscope is known in which a field of view (an oblique angle) can be freely changed by turning a prism of an optical device on a distal end to cause the prism to tilt.

### SUMMARY OF THE INVENTION

An endoscope of one aspect of the present invention is provided with: a first frame body turnably provided around a shaft body, the first frame body holding an optical device; a second frame body turnably holding the first frame body; a recessedly formed groove portion, the groove portion being in contact with an outer circumferential face of the shaft body; and an elastic member configured to give a tensile load in a direction in which the shaft body and the groove portion are in contact.

### BRIEF DESCRIPTION OF THE DRAWINGS

FIG. 1 is a perspective view showing an overall configuration of an endoscope of one aspect;

FIG. 2 is an exploded perspective view showing a configuration of a direction-of-view changing mechanism of the one aspect;

FIG. 3 is a perspective view showing the configuration of the direction-of-view changing mechanism of the one aspect;

FIG. 4 is a cross-sectional view showing a distal end portion of an insertion portion of the one aspect in which the direction-of-view changing mechanism is arranged;

FIG. 5 is a left side view showing the configuration of the direction-of-view changing mechanism of the one aspect;

FIG. 6 is an enlarged view showing a configuration of a turning shaft and an abutting portion of the one aspect;

FIG. 7 is a left side view showing a configuration of a direction-of-view changing mechanism of a first modification of the one aspect;

FIG. 8 is a left side view showing a configuration of a direction-of-view changing mechanism of a second modification of the one aspect;

FIG. 9 is a left side view showing a configuration of a direction-of-view changing mechanism of a third modification of the one aspect;

FIG. 10 is a left side view showing a configuration of a direction-of-view changing mechanism of a fourth modification of the one aspect;

FIG. 11 is a left side view showing a configuration of a direction-of-view changing mechanism of a fifth modification of the one aspect; and

FIG. 12 is a right side view showing a configuration of a direction-of-view changing mechanism according to a sixth modification of the one aspect.

### DETAILED DESCRIPTION OF THE PREFERRED EMBODIMENT(S)

A preferable embodiment of the present invention will be described below with reference to drawings. Note that, in each of drawings used in description below, a reduced scale is made different for each component so that the component is in a size recognizable on the drawing, and the present invention is not limited only to the number of components, shapes of the components, a size ratio among the components, and a relative positional relationship among the respective components illustrated on the drawings. Further, in the description below, there may be a case where description is made on an assumption that upward and downward directions relative to a figure correspond to upper and lower parts of a component, respectively.

First, an endoscope of one aspect of the present invention will be described below.

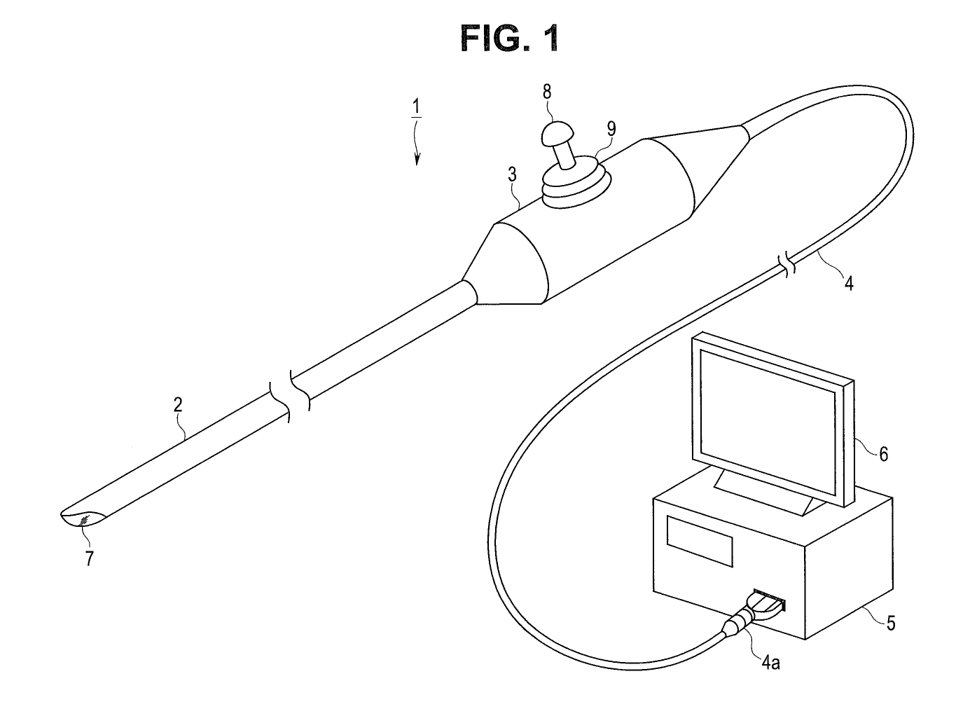

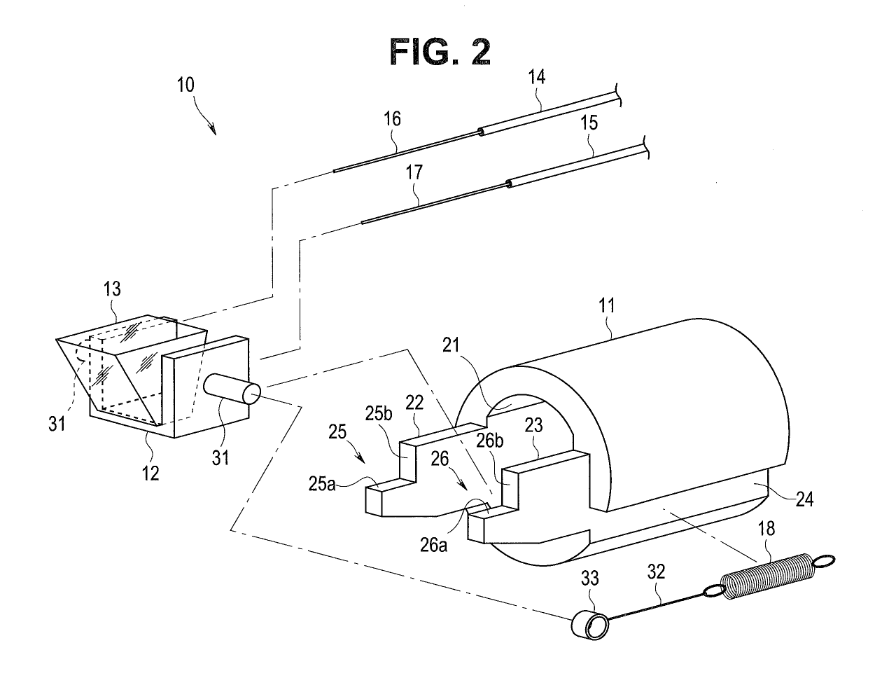

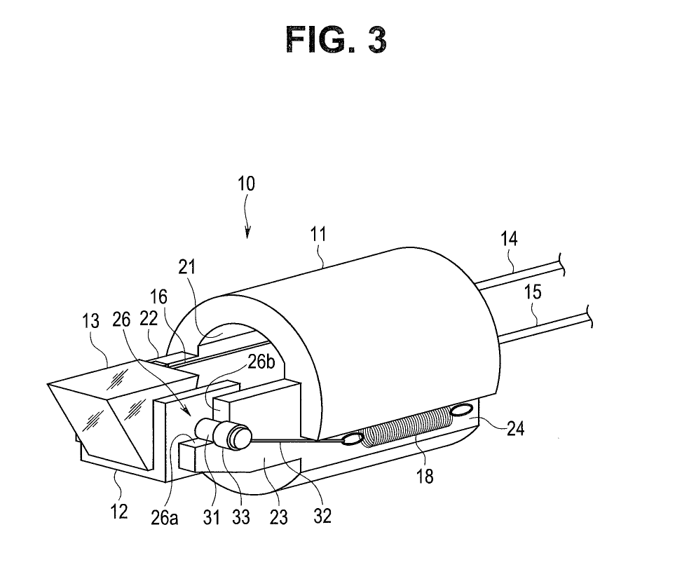

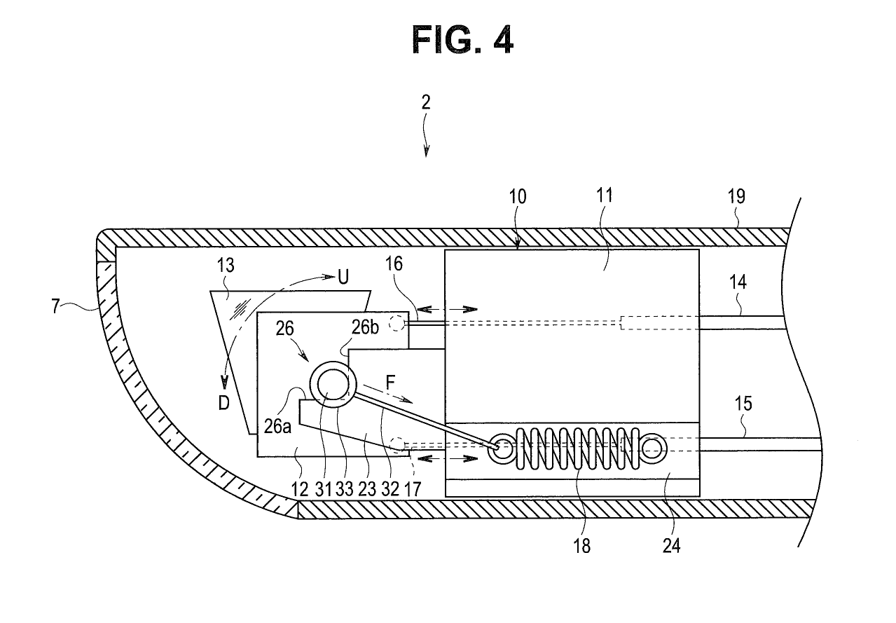

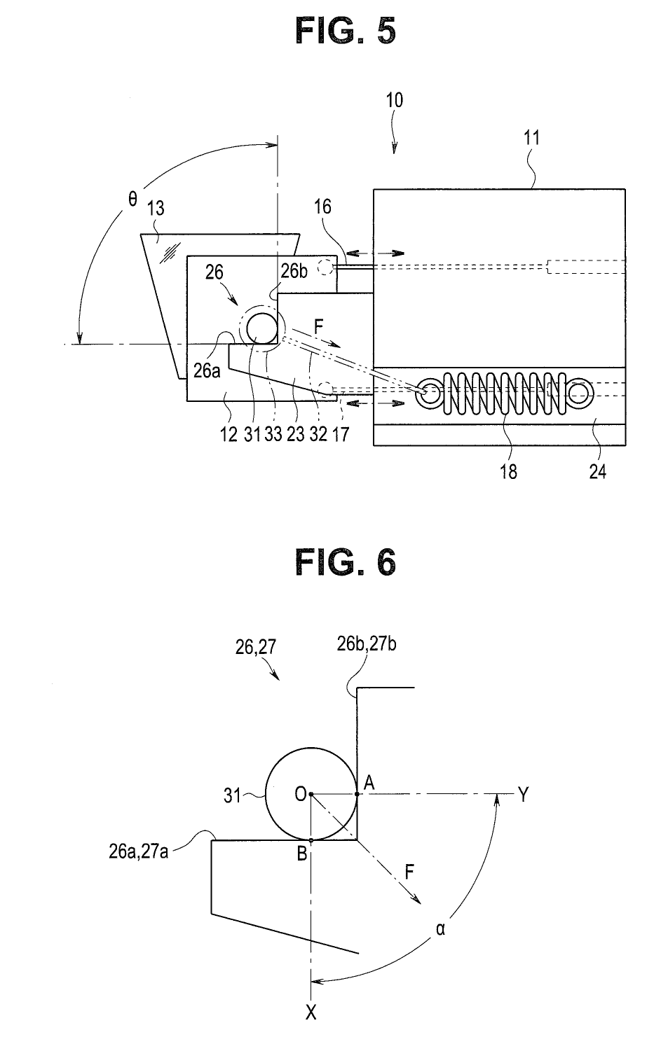

FIG. 1 is a perspective view showing an overall configuration of the endoscope; FIG. 2 is an exploded perspective view showing a configuration of a direction-of-view changing mechanism; FIG. 3 is a perspective view showing the configuration of the direction-of-view changing mechanism; FIG. 4 is a cross-sectional view showing a distal end portion of an insertion portion in which the direction-of-view changing mechanism is arranged; FIG. 5 is a left side view showing the configuration of the direction-of-view changing mechanism; and FIG. 6 is an enlarged view showing a configuration of a turning shaft and an abutting portion.

As shown in FIG. 1, an endoscope 1 of the present embodiment is a medical apparatus that can be introduced into a subject such as a human body and that is, for example, for surgery or for examining urinary organs. The endoscope 1 has a configuration for optically picking up an image of a predetermined observation region in the subject.

Note that the subject into which the endoscope 1 is introduced is not limited to a human body but may be a different living body or may be an artifact such as a machine or a structure.

The endoscope 1 is mainly configured with a rigid insertion portion 2 to be introduced into an inside of a subject/object, an operation portion 3 located at a proximal end of the insertion portion 2 and a universal cord 4 extending from a proximal end portion of the operation portion 3.

Note that the endoscope 1 here is an endoscope in a form referred to as a so-called rigid endoscope, a laparoscope, a pyeloscope or the like in which the insertion portion 2 is not provided with a part having flexibility. Of course, the configuration of the present embodiment is a technique applicable to a flexible endoscope such as an endoscope for upper endoscopy to be introduced from an oral cavity or an endoscope for lower endoscopy to be inserted from an anus.

The universal cord 4 is provided with an endoscope connector 4a to be connected to an external apparatus 5 such as a video processor, on a proximal end portion.

The external apparatus 5 is provided with an image processing portion. The image processing portion generates a video signal based on an image pickup device output signal outputted from an image pickup device not shown and outputs the video signal to an image displaying portion 6 which is a monitor. That is, in the present embodiment, an optical image (an endoscopic image) picked up by the image pickup device is displayed on the image displaying portion 6 as video.

Note that the image pickup device is a very small-sized electronic part, and a plurality of devices configured to output an electrical signal according to incident light at a predetermined timing are arrayed on a planar light receiving portion. For example, a form generally referred to as a CCD (charge-coupled device) sensor or a CMOS (complementary metal-oxide semiconductor) sensor, or other various forms are applied. The image pickup device is connected to a circuit board and the like not shown.

The insertion portion 2 of the endoscope 1 is provided with a dome-shaped cover glass 7 as an observation window on a lower part side of a distal end. On an upper part of a center of the operation portion 3 of the endoscope 1, an operation lever 8, which is an operation member of a so-called joystick type, is arranged, and a rubber boot 9, which is a cover body covering a projecting root part of the operation lever 8, is provided.

Next, a direction-of-view changing mechanism provided in the insertion portion 2 of the endoscope 1 will be described in detail below.

As shown in FIGS. 2 and 3, a direction-of-view changing mechanism 10 is configured mainly including a substantially cylindrical holding portion 11 as a second frame body, an optical device holding frame 12 as a first frame body, the optical device holding frame 12 being a recess-shaped rotating portion having an angularly U-shaped cross section, a prism 13 as an optical device held by the optical device holding frame 12, two operation wires 16 and 17 which are pulling members inserted through tube bodies 14 and 15 such as coil tubes, respectively, and a tension spring 18 as an elastic member, which is a biasing member.

The holding portion 11 is formed with metal such as stainless steel or rigid resin and has a hole portion 21 along a longitudinal axis direction. On both side parts of the holding portion 11, two arm portions 22 and 23 are provided extending toward a distal end side in a forward direction.

Moreover, on one side portion of the holding portion 11, here, on a right side portion when the holding portion 11 is seen from the distal end side, a notch portion 24 along a side face of the arm portion 23 is formed. On the two arm portions 22 and 23, abutting portions 25 and 26, which are recessedly formed V-shaped grooves, from which corner portions on an upper part side here on the distal end side are cut, are formed. The two abutting portions 25 and 26 have two planar portions 25a and 25b and two planar portions 26a and 26b, respectively, the two planar portions forming a predetermined angle.

The optical device holding frame 12 fixes and holds the prism 13 by adhesion or the like. The optical device holding frame 12 is provided with turning shafts 31, which are shaft bodies, substantially at centers of both side faces, respectively. The two turning shafts 31 are arranged on the abutting portions 25 and 26 formed on the two arm portions 22 and 23 of the holding portion 11.

Consequently, the optical device holding frame 12 is installed in a manner of freely turning around the turning shafts 31 in a state of being sandwiched between the two arm portions 22 and 23.

Note that the two turning shafts 31 may be inserted and fixed in hole portions formed on the side faces of the optical device holding frame 12 by crimping or the like or may be integrally cut and formed on the side faces of the optical device holding frame 12.

Distal ends of the two operation wires 16 and 17 extended from the tube bodies 14 and 15, respectively, are connected and fixed to upper and lower parts on a proximal end side of one side face of the optical device holding frame 12, here on the left side face by solder, caulking or the like, inserted into the hole portion 21 of the holding portion and extended from the proximal end side.

Then, the two operation wires 16 and 17 are inserted into the insertion portion 2 of the endoscope 1 in a state of being covered with the tube bodies 14 and 15 and connected to the operation lever 8 provided on the operation portion 3. Consequently, the two operation wires 16 and 17 are pulled or loosened by a forward or backward tilting operation of the operation lever 8 and move forward or backward.

The tension spring 18 is arranged on the notch portion 24 of the holding portion 11, and a proximal end of a wire 32 is connected to a hook on one end on the distal end side. A hook on the other end on a proximal end side of the tension spring 18 is hooked on a projecting portion not shown, which is provided on the notch portion 24 of the holding portion 11.

A distal end of the wire 32 is provided with a ring 33. The ring 33 is fitted onto the turning shaft 31 extending from the side face of the arm portion 23 on a right side when seen from the distal end side. Note that, instead of providing the ring 33 on the distal end of the wire 32, the distal end of the wire 32 may be formed in a ring shape to be hooked on the turning shaft 31.

Thus, tension (a tensile load) F (see FIG. 4) by a biasing force of the tension spring 18 is given to the turning shaft 31, and the turning shaft 31 on the right side when seen from the distal end side is abutted by the abutting portion 26. Furthermore, the turning shaft 31 on the left side when seen from the distal end side is abutted by the abutting portion 26 by tension which is tension (a tensile load) on the rear (proximal end) side by the two operation wires 16 and 17.

That is, the two turning shafts 31 of the optical device holding frame 12 are in a state of being pulled to a proximal-end lower side here so as to come into contact with the two planar portions 25a and 25b and the two planar portions 26a and 26b formed on the abutting portions 25 and 26 of the two arm portions 22 and 23, respectively. Consequently, the optical device holding frame 12 is turnably arranged between the two arm portions 22 and 23 in a stable state.

As shown in FIG. 4, the direction-of-view changing mechanism 10 configured as described above is arranged in a distal end portion of the insertion portion 2 of the endoscope 1. Note that the insertion portion 2 of the endoscope 1 has an exterior tube 19 which is an insertion pipe with a distal end lower part side sealed with the cover glass 7.

In the direction-of-view changing mechanism 10, when the upper operation wire 16 is pulled, and the lower operation wire 17 is loosened by an operation of the operation lever 8, the optical device holding frame 12 turns clockwise (a direction of an arrow U) around the turning shafts 31 relative to FIG. 4. Consequently, a light refraction direction of the prism 13 held by the optical device holding frame 12 changes, and the direction of view of the endoscope 1 is changed to an upper side.

On the other hand, when the lower operation wire 17 is pulled, and the upper operation wire 16 is loosened by an operation of the operation lever 8, the optical device holding frame 12 of the direction-of-view changing mechanism 10 turns counterclockwise (a direction of an arrow D) around the turning shafts 31 relative to FIG. 4. Consequently, the light refraction direction of the prism 13 held by the optical device holding frame 12 changes, and the direction of view of the endoscope

As described above, in the endoscope 1 of the present embodiment, a state in which outer circumferential faces of the turning shafts 31 of the optical device holding frame 12 holding the prism 13 which is an optical device are in contact with two positions on the two planar portions 25a and 25b and two positions on the two planar portions 26a and 26b of the abutting portions 25 and 26 formed as V-shaped grooves, which are formed on the arm portions 22 and 23 of the holding portion 11, respectively, is continuously maintained by the tension (the tensile load) F on the proximal-end lower side by the biasing force of the tension spring 18 and the tension on the rear (proximal end) side by the two operation wires 16 and 17.

Consequently, the endoscope 1 is configured such that backlash of the optical device holding frame 12 that turns, holding the prism 13 which is an optical device does not occur, and highly accurate positioning of the prism 13 is easily performed.

Moreover, at the time of assembling the direction-of-view changing mechanism 10, the optical device holding frame 12 is easily positioned at a predetermined position only by adding a biasing force by the tension spring 18 and tension of the two operation wires 16 and 17 so that the outer circumferential faces of the turning shafts 31 are abutted by the two planar portions 25a and 25b and the two planar portions 26a and 26b of the abutting portions 25 and 26, respectively, after installing the turning shafts 31 of the optical device holding frame 12 on the abutting portions 25 and 26 formed as V-shaped grooves. Therefore, assembly workability is improved.

Note that, as shown in FIG. 5, an angle θ formed by the two planar portions 26a and 26b (25a and 25b) of the abutting portion 26 (25) is only required to be 180° or smaller, and the angle is shown as substantially 90° here (in FIG. 5, the two planar portions 26a and 26b of the abutting portion 26 of the right side face are shown).

As shown in FIG. 6, a direction of the tension (the tensile load) F by the tension spring 18 is only required to be included in an area a between imaginary lines X and Y that pass points of contact A and B at which the left (right) turning shaft 31 is in contact with the two planar portions 25a and 25b (26a and 26b) of the abutting portion 25 (26) from a center O of the turning shaft 31, and such a direction that the area a is equally divided into two is desirable.

### MODIFICATIONS

Note that configurations of other aspects having operation and effects similar to the operation and effects of the endoscope 1 of the embodiment described above will be illustrated in various modifications below. In the configuration of the above embodiment and the configurations of the various modifications below, main portions of the embodiment and the modifications can be combined.

### First Modification

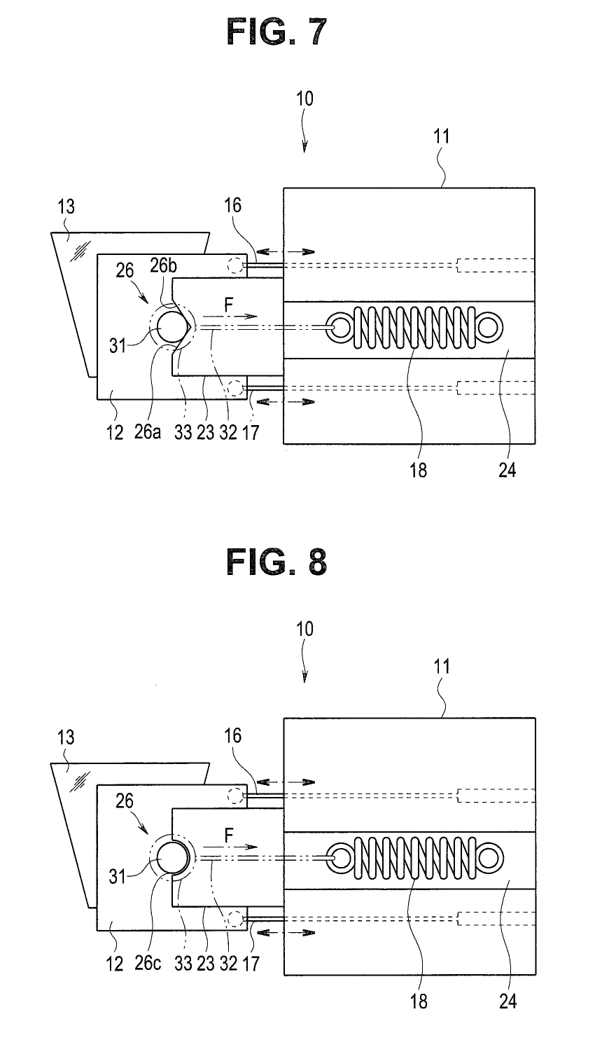

FIG. 7 is a right side view showing a configuration of a direction-of-view changing mechanism according to a first modification.

As shown in FIG. 7, a configuration is also possible in which the abutting portion 26 (25) configured with the two planar portions 26a and 26b (25a and 25b) are formed as a V-shaped groove recessedly formed on the distal end face of the arm portion 22 (23), and the tension spring 18 is arranged so that a biasing force is given toward the proximal end side in a backward direction, and the tension (the tensile load) F is applied to the turning shaft 31 on the right toward the proximal end side (in FIG. 7, the two planar portions 26a and 26b of the abutting portion 26 on the right side face are shown).

### Second Modification

FIG. 8 is a right side view showing a configuration of a direction-of-view changing mechanism according to a second modification.

As shown in FIG. 8, a configuration is also possible in which the abutting portion 26 (25) is formed as a curved face 26c (25c) on the distal end face of the arm portion 22 (23) to be in an arc groove shape recessedly formed in an R shape substantially same as the outer circumferential faces of the turning shafts 31, and the tension spring 18 is arranged so that a biasing force is given to the proximal end side in the backward direction, and the tension (the tensile load) F is applied to the turning shaft 31 on the right toward the proximal end side (in FIG. 8, only the curved face 26c of the abutting portion 26 on the right side face is shown).

### Third Modification

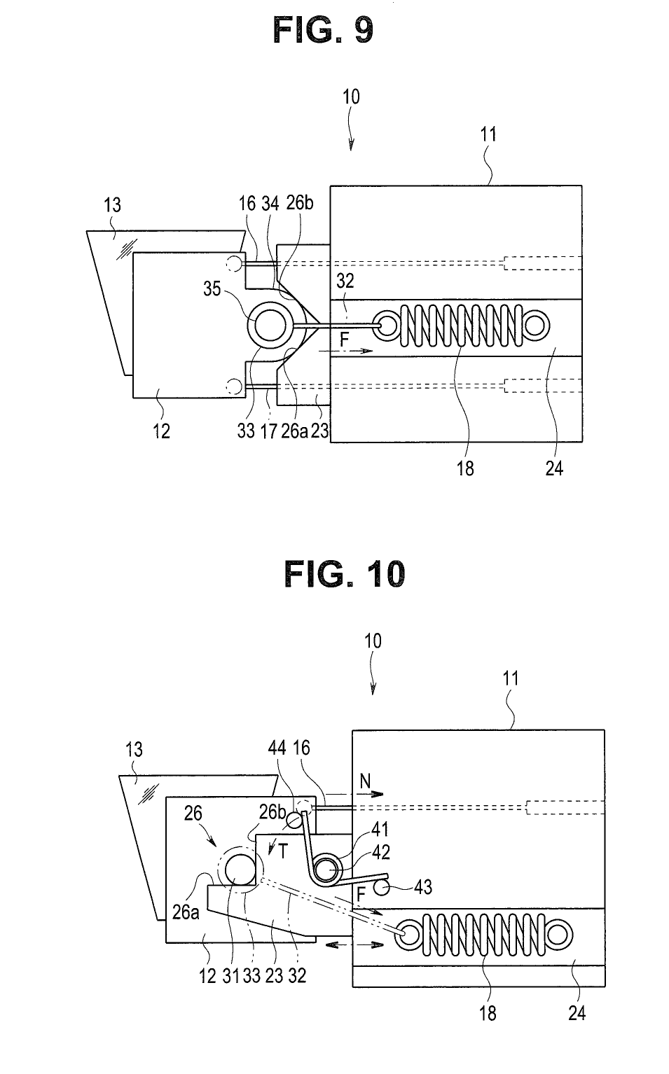

FIG. 9 is a right side view showing a configuration of a direction-of-view changing mechanism according to a third modification.

As shown in FIG. 9, a configuration is also possible in which, instead of providing the turning shafts 31, a proximal end of the optical device holding frame 12 is formed as an arc-shaped curved face 34, and the curved face 34 is abutted by the two planar portions 26a and 26b (25a and 25b) of the abutting portion 26 (25) that is recessedly formed as a V-shape groove on the distal end face of the arm portion 22 (23) (in FIG. 9, the two planar portions 26a and 26b of the abutting portion 26 on the right side face are shown).

Note that, here, a projecting portion 35 configured for the ring 33 to be hooked is provided on a proximal end part of the optical device holding frame 12, and the tension spring 18 is arranged so that a biasing force is given to the proximal end side in the backward direction, and the tension (the tensile load) F is applied to the optical device holding frame 12 toward the proximal end side.

### Fourth Modification

FIG. 10 is a right side view showing a configuration of a direction-of-view changing mechanism according to a fourth modification.

As shown in FIG. 10, a torsion spring 41, which is a biasing member configured to bias the turning shafts 31 counterclockwise here and generate rotation torque T to bias the optical device holding frame 12 counterclockwise around the turning shafts 31 here to change the direction of view downward, may be provided, and only the operation wire 16 to perform a pulling operation of the optical device holding frame 12 clockwise around the turning shafts 31 here to change the direction of view upward may be provided (in FIG. 10, the two planar portions 26a and 26b of the abutting portion 26 on the right side face are shown).

Note that the torsion spring 41 is fitted to a spring support shaft 42 provided on the arm portion 23, and both ends are in contact with projecting portions 43 and 44 provided on the holding portion 11 and the optical device holding frame 12.

That is, here, by the operation wire 16 being pulled, tension N of the operation wire 16 exceeds the rotational torque T of the torsion spring 41, and the optical device holding frame 12 turns clockwise around the turning shafts 31. Then, by the operation wire 16 being loosened, the rotational torque T of the torsion spring 41 exceeds the tension N of the operation wire 16, and the optical device holding frame 12 turns counterclockwise around the turning shafts 31.

According to such a configuration, it is possible to turn the optical device holding frame 12 only by a pulling/loosening operation of the one operation wire 16 and change the direction of view of the endoscope 1 upward or downward.

Note that even though a direction of resultant force of the tension N of the operation wire 16 applied to the turning shaft 31 on the left and the rotational torque T of the torsion spring 41 is away from (floats from) the abutting portion 26 (25), the turning shaft 31 on the right (left) can be stably abutted by the two planar portions 26a and 26b (25a and 25b) of the abutting portion 26 (25) by the tension (the tensile load) F by the tension spring 18.

### Fifth Modification

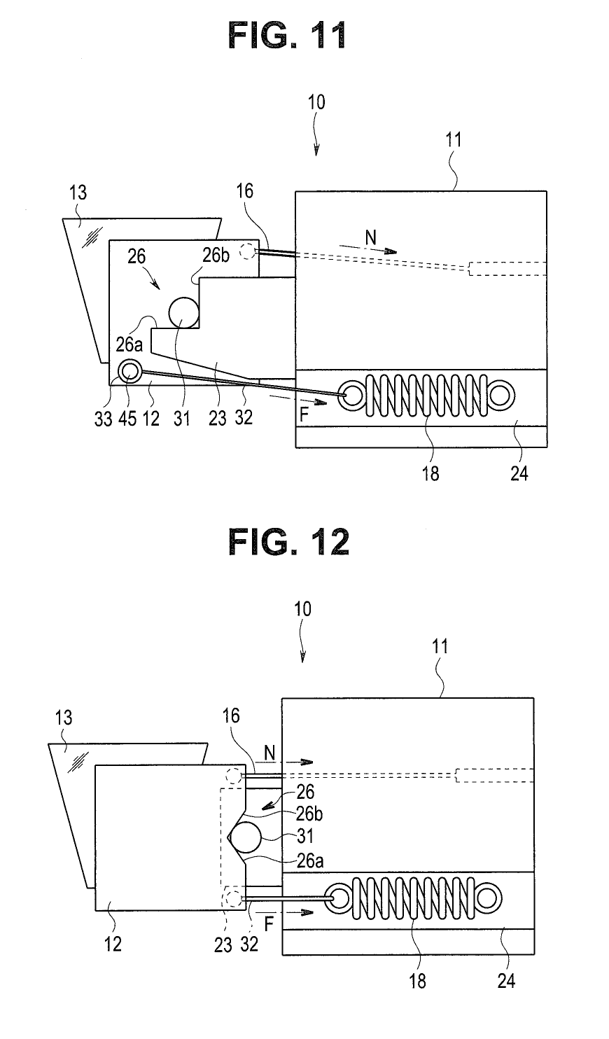

FIG. 11 is a right side view showing a configuration of a direction-of-view changing mechanism according to a fifth modification.

As shown in FIG. 11, here, a projecting portion 45 configured so that the ring 33 is hooked is provided on a distal end lower part of the optical device holding frame 12, and the tension spring 18 is arranged so that a biasing force is given to the proximal end lower side, and the tension (the tensile load) F is applied to the optical device holding frame 12 toward the proximal end lower side.

Moreover, here, the tension N by the operation wire 16 is also set to be applied to the optical device holding frame 12 toward the proximal end lower side.

That is, here, by the operation wire 16 being pulled, the tension N of the operation wire 16 exceeds the tension (the tensile load) F of the tension spring 18, and the optical device holding frame 12 turns clockwise around the turning shafts 31. Then, by the operation wire 16 being loosened, the tension (the tensile load) F of the tension spring 18 exceeds the tension N of the operation wire 16, and the optical device holding frame 12 turns counterclockwise around the turning shafts 31.

Even if such a configuration is adopted, it is also possible to turn the optical device holding frame 12 only by a pulling/loosening operation of the one operation wire 16 and change the direction of view of the endoscope 1 upward or downward.

Note that it is necessary that the resultant force of the tension N of the operation wire 16 applied to the turning shaft 31 on the left and the tension (the tensile load) F of the tension spring 18 continuously works in the direction of causing the turning shaft 31 on the right (left) to abut against the two planar portions 26a and 26b (25a and 25b) of the abutting portion 26 (25). Therefore, a configuration is illustrated here in which the tension N of the operation wire 16 and the tension (the tensile load) F of the tension spring 18 work in a direction of pulling the optical device holding frame 12 toward the proximal end lower side.

That is, since it is only required that the resultant force of the tension N of the operation wire 16 and the tension (the tensile load) F of the tension spring 18 works in the direction of causing the turning shaft 31 on the right (left) to abut against the two planar portions 26a and 26b (25a and 25b) of the abutting portion 26 (25), a configuration is also possible in which the tension N of the operation wire 16 or the tension (the tensile load) F of the tension spring 18 works in the upper side in a proximal end direction if the above condition is satisfied.

### Sixth Modification

FIG. 12 is a right side view showing a configuration of a direction-of-view changing mechanism according to a sixth modification.

In the embodiment and each modification described above, a configuration has been illustrated in which the turning shafts 31 are provided on the optical device holding frame 12, and the abutting portion 26 (25) configured with the two planar portions 26a, 26b (25a, 25b) are formed on the arm portion 23 (22). However, for example, as shown in FIG. 12, a configuration is also possible in which the abutting portion 26 (25) configured with the two planar portions 26a, 26b (25a, 25b) is formed on the proximal end side of the optical device holding frame 12, and the turning shaft 31 on the right (left) is provided on the aim portion 23 (22) (in FIG. 12, the two planar portions 26a and 26b of the abutting portion 26 on the right side face are shown).

Note that, here, a configuration is adopted in which the abutting portion 26 (25) configured with the two planar portions 26a and 26b (25a and 25b) is a recessedly formed V-shaped groove similar to the V-shaped groove of the first modification, and the tension spring 18 is arranged so that a biasing force is given to the proximal end side in the backward direction, and the tension (the tensile load) F is applied to the proximal end lower side of the optical device holding frame 12 toward the proximal end side.

Similarly to the fifth modification, the operation wire 16 is connected to the proximal end upper side of the optical device holding frame 12 here, and a state is shown in which the tension N by the operation wire 16 and the tension (the tensile load) F by the tension spring 18 are balanced.

Note that, instead of the abutting portion 26 (25) as a V-shaped groove configured with the two planar portions 26a and 26b (25a and 25b), the optical device holding frame 12 may be formed as a curved face which is in an arc groove shape recessedly formed in an R shape almost similar to the outer circumferential faces of the turning shafts 31, similar to the second modification.

Note that though the tension spring 18 is used to cause the tension (the tensile load) F to be generated, in the embodiment and modifications described above, means for generating pulling force by another elastic member made of rubber or the like may be used instead.

Moreover, though an electronic endoscope provided with an image pickup device has been illustrated as the endoscope 1, the endoscope 1 is not limited to an electronic endoscope. Each of the embodiment and modifications described above can be applied to a configuration in which a relay lens is provided to transfer a subject/object image.

The invention described in each of the embodiment and modifications described above is not limited to the embodiment and the modifications, but, additionally, various alterations can be performed at a stage of practicing the invention, within a range not departing from the spirit of the invention. Moreover, each of the embodiment and modifications described above includes inventions at various stages, and various inventions can be extracted by appropriately combining a plurality of disclosed constituent features.

For example, even if some constituent features are deleted from all constituent features shown in each of the embodiment and modifications, a configuration obtained after deleting the constituent features can be extracted as an invention if the stated problem can be solved, and the stated effects can be obtained.

According to the present invention, it is possible to provide an endoscope in which highly accurate positioning of an optical device to change a direction of view can be performed without backlash, and for which assembly workability is improved.

What is claimed is:

1. An endoscope comprising:
a first frame body turnably provided around a shaft body, the first frame body holding an optical device;
a second frame body turnably holding the first frame body;
a recessedly formed groove portion, the groove portion being in contact with an outer circumferential face of the shaft body; and
an elastic member configured to give a tensile load in a direction in which the shaft body and the groove portion are in contact.

2. The endoscope according to claim 1, wherein
the shaft body is provided in the first frame body, and
the groove portion is formed on the second frame body.

3. The endoscope according to claim 1, wherein the groove portion includes two planes forming a predetermined angle, the outer circumferential face of the shaft body being in contact with the two planes.

4. The endoscope according to claim 3, wherein the elastic member is a first biasing member configured to give the tensile load in a direction in which the shaft body is in contact with the two planes.

5. The endoscope according to claim 1, comprising a pulling member configured to perform a turning operation of the first frame body and give tension to the shaft body in the direction of being in contact with the groove portion.

6. The endoscope according to claim 1, comprising a pulling member configured to perform a turning operation of the first frame body to give tension to the shaft body, wherein
resultant force of the tension and a tensile load by the elastic member works in a direction of being in contact with the groove portion.

7. The endoscope according to claim 5, comprising a second biasing member configured to give rotational torque to the first frame body in another direction opposite to one direction in which the first frame body turns around the shaft body by the pulling member being pulled.

8. The endoscope according to claim 7, wherein the second biasing member is a torsion spring.

9. The endoscope according to claim 5, wherein the pulling member is an operation wire.

10. The endoscope according to claim 1, wherein the elastic member is a tension spring.
